(19) United States
(12) Patent Application Publication
(10) Pub. No.: US 2015/0018560 A1
(43) Pub. Date: Jan. 15, 2015

(54) Process for preparing cis-5-[1-(4-chlorophenyl)-methylene]-1-hydroxymethyl-2,2-dimethylcyclopentanol

(71) Applicant: BASF AGRO B.V., ARNHEM (NL), ZÜRICH-BRANCH, Zürich (CH)

(72) Inventors: Thomas Zierke, Bohl-lggelheim (DE); Paul Kemper, Dudenhofen (DE)

(73) Assignee: BASf ARGO B.V. ARNHEM (NL). ZURICH-BRANCH, Zurich (CH)

(21) Appl. No.: 14/377,720

(22) PCT Filed: Feb. 7, 2013
(86) PCT No.: PCT/EP2013/052402

§ 371 Date: Aug. 8, 2014

(30) Foreign Application Priority Data
Feb. 10, 2012 (EP) 12154970.3

(51) Int. Cl. C07C 29/132; C07D 405/06; C07D 303/32

(52) U.S. Cl. CPC C07C 29/132; C07D 303/32; C07D 405/06
USPC 548/268.8; 568/807; 549/546

(57) ABSTRACT

The present invention relates to a process for preparing a diol of the formulae (Ia) or (Ib), or a mixture thereof,

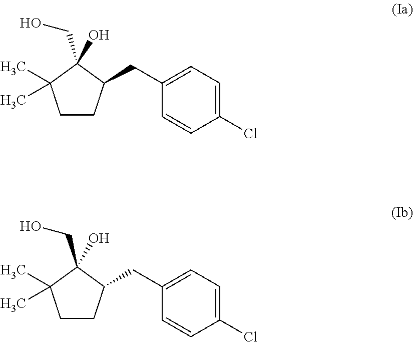 [SMILES: CC1(C)CC[C@H](Cc2ccc(Cl)cc2)[C@@]1(O)CO ; CC1(C)CC[C@@H](Cc2ccc(Cl)cc2)[C@]1(O)CO]

comprising the reaction of an epoxy alcohol of formulae (Va) or (Vb), or a mixture thereof,

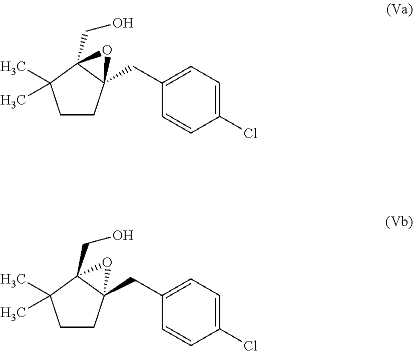 [SMILES: CC1(C)CC[C@]2(Cc3ccc(Cl)cc3)O[C@]12CO ; CC1(C)CC[C@@]2(Cc3ccc(Cl)cc3)O[C@@]12CO]

with alkali metal borohydride or earth alkaline borohydride, in particular sodium borohydride, and anhydrous aluminum(III) chloride.

The invention also relates to processes and intermediates for preparing an epoxy alcohol of formulae (Va) or (Vb). The invention furthermore relates to processes for preparing cis-metconazole in which a diol of the formulae (Ia) or (Ib) is applied as a precursor.

The present invention relates to a process for preparing cis-5-(4-chlorobenzyl)-1-hydroxymethyl-2,2-dimethylcyclopentanol of the formulae (Ia) or (Ib), hereinafter also termed as cis-diol of the formulae (Ia) or (Ib), or a mixture of these diols, such as a racemic mixture.

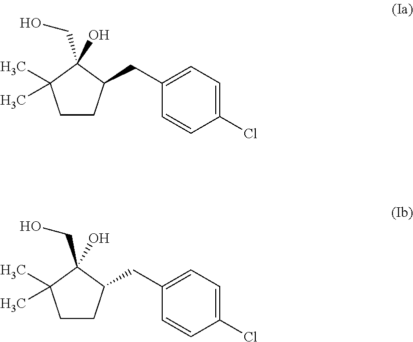 [SMILES: CC1(C)CC[C@H](Cc2ccc(Cl)cc2)[C@@]1(O)CO ; CC1(C)CC[C@@H](Cc2ccc(Cl)cc2)[C@]1(O)CO]

The enantiomers of cis-5-(4-chlorobenzyl)-1-hydroxymethyl-2,2-dimethylcyclopentanol of formulae (Ia) and (Ib) are valuable as precursors for the preparation of the (−)-cis-metconazole ((1S,5R)-5-(4-chlorobenzyl)-2,2-dimethyl-1-(1H-1,2,4-triazol-1-ylmethyl)cyclopentanol) and (+)-cis-metconazole ((1R,5S)-5-(4-chlorobenzyl)-2,2-dimethyl-1-(1H-1,2,4-triazol-1-ylmethyl)cyclopentanol), respectively, and, in the form of their mixtures, for the preparation of the racemic cis-metconazole cis-((1RS;5SR)-5-(4-chlorobenzyl)-2,2-dimethyl-1-(1H-1,2,4-triazol-1-ylmethyl)cyclopentanol).

EP-A 359305 suggests the preparation of a racemic mixture of the diols of the formulae (Ia) and (Ib) by reacting either the racemic [5-(4-chlorobenzyl)-2,2-dimethyl-6-oxabicyclo[3.1.0]-cyclohex-1-yl]methanol of formula (V), hereinafter also termed epoxide (V), or the corresponding acid or ester of formula (V),

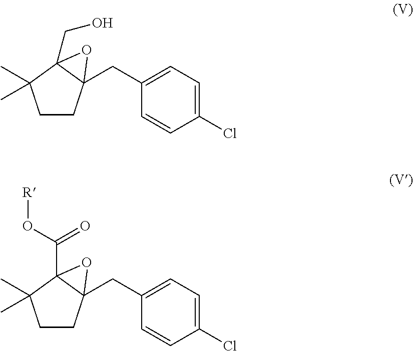

wherein R′ represents a hydrogen atom, an alkyl group or a cycloalkyl group, with a reducing agent, preferably a complex metal hydride, e.g. a mixture of lithium aluminum hydride (LiAlH₄) and aluminum(III) chloride. The use of LiAlH₄ suffers, however, from being hazardous and expensive.

EP-A 474303 discloses a process for preparing a racemic mixture of the diols of (Ia) and (Ib) starting from 1-(4-chlorobenzyl)-4,4-dimethyl-cyclohex-1-en-3-one of formula (XI).

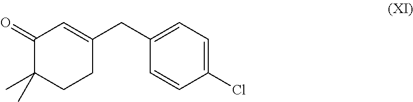 [SMILES: CC1(C)CCC(Cc2ccc(Cl)cc2)=CC1=O]

However this process does not provide access to either of the diol enantiomers (Ia) or (Ib), without resorting to cumbersome racemate resolution techniques as additional steps.

EP 488396 discloses a process for preparing optically active (−)-cis or (+)-cis-metconazole, which process comprises preparation of the [(1S,5R)-5-(4-chlorobenzyl)-2,2-dimethyl-6-oxabicyclo[3.1.0]-cyclohex-1-yl]methanol of the formula (Va) or its enantiomer [(1R,5S)-5-(4-chlorobenzyl)-2,2-dimethyl-6-oxabicyclo[3.1.0]-cyclohex-1-yl]methanol of the formula (Vb), hereinafter termed epoxyalcohol (Va) or (Vb), by stereoselective epoxidation of the prochiral[5-(4-chlorobenzyl)-2,2-dimethylcyclohex-1-enyl]methanol of the formula (IV), hereinafter termed allylalcohole (IV), according to a Sharpless expoxidation in the presence of either L-(+) diethyltartrate or D-(−)diethyltartrate as chiral auxiliary.

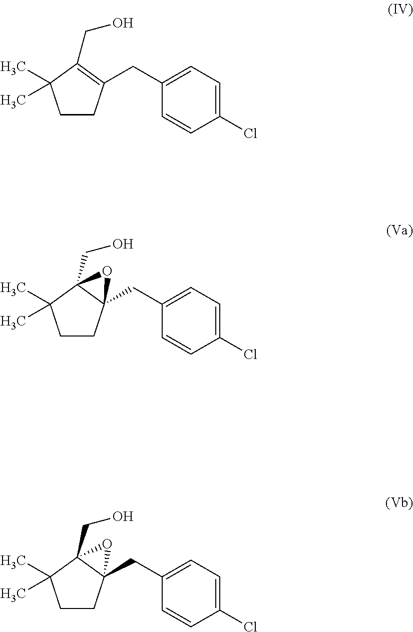 [SMILES: CC1(C)CCC(Cc2ccc(Cl)cc2)=C1CO ; CC1(C)CC[C@]2(Cc3ccc(Cl)cc3)O[C@]12CO ; CC1(C)CC[C@@]2(Cc3ccc(Cl)cc3)O[C@@]12CO]

According to EP 488396 the epoxyalcohols of formulae (Va) or (Vb) are then reacted with an alkylsulfonyl chloride or a phenylsulfonylchloride, thereby converting the OH-group of Va or Vb, respectively, into a sulfonic ester group. The sulfonic ester is then reacted with an azol, e.g. 1,2,4 triazol. The thus obtained triazolylmethyloxabicyclohexane derivatives of formulae (Xa) or (Xb) are reduced with lithium aluminium hydride in the presence of aluminium(III)chloride to obtain the (−)-cis- or (+)-cis-metconazole, respectively.

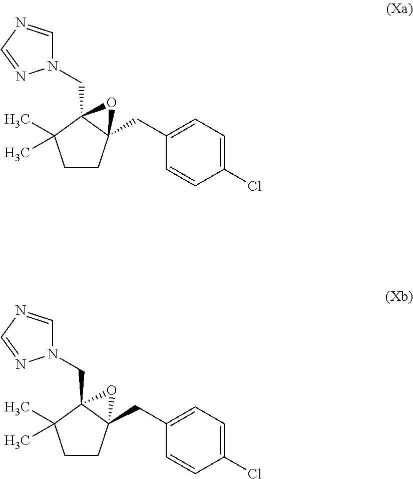 [SMILES: CC1(C)CC[C@]2(Cc3ccc(Cl)cc3)O[C@]12Cn1cncn1 ; CC1(C)CC[C@@]2(Cc3ccc(Cl)cc3)O[C@@]12Cn1cncn1]

The process of EP 488396 still suffers from the use of hazardous and expensive lithium aluminum hydride. Moreover, the products of formulae (Xa) and (Xa), as well as their mixtures, require chromatographic purification. The process of EP 488396 also suffers from the low yield of the final reduction step resulting in a substantial loss of the enantioselectively prepared precursor of formulae (Xa) or (Xb), which is accessible only via a extensive synthesis procedure.

It is the object of the present invention to provide an economically attractive and technically feasible process that allows the preparation of the cis-diols Ia and Ib, either in the form of their racemate or in form of the separate enantiomers, starting from readily available starting materials. The process should be easy to perform and be suitable for industrial scale production. In addition, it should be inexpensive and not require highly hazardous reagents, such as LiAlH₄.

The object is achieved by the processes described in detail below.

The present invention provides a process for preparing the cis-diols of the formulae (Ia) or (Ib), or a mixture thereof, comprising the reaction of an epoxy alcohol of formulae (Va) or (Vb), or a mixture thereof, e.g. a racemic mixture (i.e. the compound of formula (V)),

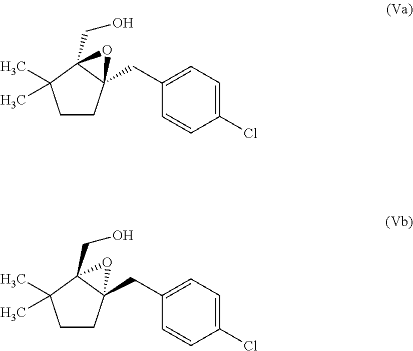 [SMILES: CC1(C)CC[C@]2(Cc3ccc(Cl)cc3)O[C@]12CO ; CC1(C)CC[C@@]2(Cc3ccc(Cl)cc3)O[C@@]12CO]

with alkali metal borohydride or earth alkaline borohydride, in particular sodium borohydride, and anhydrous aluminum(III) chloride. This process is hereinafter also referred to as “process A”.

The process provides the compounds of formulae (Ia) and (Ib) in high yield with high stereoselectivity. The ratio of the cis-forms (Ia) and (Ib) to the corresponding trans diastereomers cis:trans is usually >90:10. Furthermore, the process avoids the use of highly hazardous reagents, such as LiAlH₄ thereby rendering the process more feasible and economic. If the enantiomer of the formula (Va) is used in process A, the enantiomer of formula (Ia) will be obtained with high enantiomeric excess while the enantiomer of formula (Ib) will be obtained with high enantiomeric excess, if the enantiomer of the formula (Vb) is used as a starting material is used in process A. The enantiomeric excess (ee) of the compound of formulae (Ia) and (Ib), respectively, will depend on the (ee) value of the starting material used, but it will generally be higher than 80% ee, if the (ee) value of the compound of formulae (Va) or (Vb) is higher than 80%.

In the context of the present invention, the terms used generically are defined as follows:

The prefix Cₓ-C_{y} denotes the number of possible carbon atoms in the particular case.

The term “halogen” denotes in each case fluorine, bromine, chlorine or iodine, especially chlorine or bromine.

The term “C₁-C₄-alkyl” denotes a linear or branched alkyl radical comprising from 1 to 4 carbon atoms, such as methyl, ethyl, propyl, 1-methylethyl(isopropyl), butyl, 1-methylpropyl(sec-butyl), 2-methylpropyl(isobutyl) or 1,1-dimethylethyl(tert-butyl).

The term “C₁-C₄-haloalkyl”, as used herein and in the haloalkyl units of C₁-C₄-haloalkoxy, describes straight-chain or branched alkyl groups having from 1 to 4 carbon atoms, where some or all of the hydrogen atoms of these groups have been replaced by halogen atoms. Examples thereof are chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 3,3,3-trifluoroprop-1-yl, 1,1,1-trifluoroprop-2-yl, 3,3,3-trichloroprop-1-yl, heptafluoroisopropyl, 1-chlorobutyl, 2-chlorobutyl, 3-chlorobutyl, 4-chlorobutyl, 1-fluorobutyl, 2-fluorobutyl, 3-fluorobutyl, 4-fluorobutyl and the like.

The enantiomeric excess is commonly defined as the ratio

[W(a)−W(b)]:[W(a)+W(b)]

where W(a) is the amount of enantiomer a, e.g. the compound of formula (Ia) or the compound of formula (Va), and W(b) is the amount of enantiomer b, e.g. the compound of formula (Ib) or the compound of formula (Vb).

The reactions of the invention as described hereinafter are performed in reaction vessels customary for such reactions, the reaction being carried out in a continuous, semi-continuous or batchwise manner. In general, the particular reactions will be carried out under atmospheric pressure. The reactions may, however, also be carried out under reduced or elevated pressure.

The reaction of process A according to the invention for preparing a cis-diol of the formulae (Ia) or (Ib), or a mixture thereof may be regarded as a reductive epoxide ring opening. The conversion is effected by reacting either the epoxy alcohol of formula (Va) or its enantiomer of formula (Vb) or a mixture of the enantiomers (Va) and (Vb), in particular a racemic mixture thereof, with an alkali metal or alkaline earth metal borohydride and anhydrous aluminum(III) chloride.

Preferred borohydrides for the transformation of process A are alkali metal borohydrides, such as sodium borohydride and potassium borohydride. A particular preferred metal borohydride is sodium borohydride.

In the reaction of process A the borohydride, in particular the alkali metal borohydride, especially sodium borohydride, is preferably used in an amount of 0.8 to 2.0 mol, more preferably of 1.0 to 1.7 mol, even more preferably of 1.2 to 1.5 mol and especially of 1.3 to 1.4 mol, based in each case on 1 mol of the epoxy alcohol (Va) or (Vb), or the mixture thereof.

In the reaction of process A the aluminum(III) chloride is preferably used in an amount of 0.2 to 1.5 mol, more preferably of 0.4 to 1.1 mol and especially of 0.6 to 0.9 mol, based in each case on 1 mol of the epoxy alcohol (Va) or (Vb), or the mixture thereof.

The molar ratio of the borohydride, in particular the alkali metal borohydride, especially sodium borohydride, to the aluminum(III) chloride is preferably from 0.8:1 to 4.0:1, especially from 1.4:1 to 3.0:1.

The reaction of process A is preferably carried out in an organic solvent.

It has generally been found to be advantageous to use an aprotic organic solvent for the reaction of process A. Useful aprotic organic solvents here include ethers, for example, aliphatic C₃-C₁₀-ethers having 1, 2, 3, or 4 oxygen atoms, such as ethylene glycol dimethyl ether (glyme), diethylene glycol dimethyl ether (diglyme), triethylene glycol dimethyl ether (triglyme), diethyl ether, dipropyl ether, methyl isobutyl ether, tert-butyl methyl ether and tert-butyl ethyl ether, alicyclic C₄-C₆-ethers, such as tetrahydrofuran (THF), tetrahydropyran, 2-methyltetrahydrofuran, 3-methyltetrahydrofuran and 1,4-dioxane, aromatic hydrocarbons, such as benzene, toluene, the xylenes and mesitylene, or mixtures of these solvents with one another.

The solvent for the reaction of process A is preferably selected from ethers, in particular aliphatic C₃-C₁₀-ethers, alicyclic C₃-C₆-ethers and mixtures thereof, and mixtures of ethers with aliphatic or aromatic hydrocarbons and more preferably from aliphatic C₄-C₁₀-ethers having 2, 3, or 4 oxygen atoms and alicyclic C₄-C₆-ethers, such as glyme, diglyme, triglyme, THF or 1,4-dioxane, or mixtures thereof. In this context C₄-C₁₀-ethers having 2, 3, or 4 oxygen atoms, especially glyme, diglyme and triglyme and mixtures thereof are particularly preferred solvents.

The total amount of the solvent used in the reaction of process A according to the invention is typically in the range from 1000 to 5000 g and preferably in the range from 1500 to 3000 g, based on 1 mol of the epoxy alcohol Va or Vb, or the mixture thereof.

Preference is given to using solvents which are essentially anhydrous, i.e. have a water content of less than 1000 ppm and especially not more than 200 ppm.

The reactants can in principle be contacted with one another in any desired sequence. For example, the borohydride and the aluminum(III) chloride, if appropriate in dissolved or dispersed form, can be initially charged and mixed with each other. The obtained mixture can then be admixed with the epoxy alcohol of formulae (Va) or (Vb), or a mixture thereof. Conversely, the epoxy alcohol of formulae (Va) or (Vb), or a mixture thereof, if appropriate in dissolved or dispersed form, can be initially charged and admixed with a mixture of the borohydride and the aluminum(III) chloride. Alternatively, all reactants can also be added simultaneously to the reaction vessel. As an alternative to the joint addition of the borohydride and aluminum(III) chloride they can also be added separately to the reaction vessel. Both of them can independently of one another be added, either in a solvent or in bulk, before or after the addition of the epoxy alcohol (Va) or (Vb), or a mixture thereof, as long as aluminum(III) chloride is not contacted with the epoxy alcohol (Va) or (Vb) in the absence of the borohydride.

It has been found to be beneficial to initially charge the reaction vessel with a mixture of the borohydride and aluminum(III) chloride, e.g. in dispersed form or preferably in dissolved form, and then to add the epoxy alcohol (Va) or (Vb), or a mixture thereof. The epoxy alcohol (Va) or (Vb), or a mixture thereof, is employed as such or in dissolved form.

In general, the reaction of process A is performed under temperature control. The reaction is typically effected in a closed or preferably in an open reaction vessel with stirring apparatus. The reaction temperature of reaction of process A depends on different factors, in particular on the reactivity of the borohydride used, and can be determined by the person skilled in the art in the individual case, for example by simple preliminary tests. In general, the conversion of process A is performed at a temperature in the range from −20 to 100° C., preferably in the range from −10 to 80° C., more preferably in the range from −5 to 70° C. and specifically in the range from 0 to 50° C.

According to a preferred embodiment of the invention the reaction of process A is initiated at a lower temperature, for instance −20° C., preferably −10° C., more preferably −5° C. and especially 0° C., and the temperature is then increased stepwise or continuously increased to an upper temperature of for instance 100° C., preferably 80° C., more preferably 70° C. and especially 50° C.

Depending on the solvent used, the reaction temperature and on whether the reaction vessel possesses a vent, a pressure of generally 1 to 5 bar and preferably of 1 to 3 bar is established during the reaction.

The work-up of the reaction mixtures obtained in the reaction of process A and the isolation of the cis diol of the formulae (Ia) or (Ib), or a mixture thereof, are effected in a customary manner, for example by an aqueous extractive work-up or by removing the solvent, for example under reduced pressure. Generally, cis diol compounds of the formulae (Ia) or (Ib), as well as the mixtures thereof, are obtained in sufficient purity by applying such measures or a combination thereof. Thus, additional purification steps, in particular elaborated ones such as chromatography or distillation are usually not necessary. If desired, however, further purification can be effected by methods commonly used in the art.

Preferably the reaction mixture obtained in the reaction of process A, for work-up, is concentrated by removing all or most of the solvent and then the residue is treated either simultaneously or successively with a suitable aprotic organic solvent being insoluble or only slightly soluble in water, such as toluene, and an aqueous basic solution, such as aqueous sodium hydroxide, preferably at an elevated temperature of about 25 to 55° C. Optionally, after removal of the aqueous phase, the organic phase is treated again with the aforementioned aqueous basic solution. As an alternative or in addition to the aforementioned basic treatment the organic phase can also be extracted at least once with an aqueous acidic solution, such as an aqueous solution of sulfuric acid, e.g. 10% sulfuric acid, and preferably afterwards washed with an aqueous solution having a neutral or approximately neutral pH value. The organic phase containing the cis diol (Ia) or (Ib) can then be introduced into a further reaction step, either directly or after partial or complete removal of the solvent. Alternatively, the organic phase is concentrated and the crude product thus obtained is subsequently retained for uses or sent directly to a use, for example used in a further reaction step, or be purified further beforehand. Preferably, the organic phase obtained after one or more treatments with an aqueous basic solution is introduced directly or after partial concentration into a further reaction step.

The starting materials for process A, namely the compounds of formulae (Va) or (Vb) can be prepared by analogy to the process of EP 488396 by epoxidation of the allylic alcohol (IV), which itself can be prepared from 7-(4-chloro-benzyl)-4,4-dimethyl-1-oxa-spiro[2.4]heptane of the formula (IX) according to the method described in EP 488396.

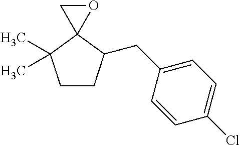 [SMILES: CC1(C)CCC(Cc2ccc(Cl)cc2)C12CO2]

It has surprisingly be found that the starting compounds of process A, namely the epoxyalcohols of the formulae (Va) or (Vb), as well as their mixtures, can also be prepared starting from the 2-(4-chlorobenzyl)-5,5-dimethylcyclopent-1-enecarbaldehyde, i.e. the compound of the formula (III),

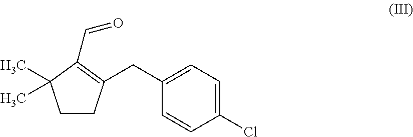 [SMILES: CC1(C)CCC(Cc2ccc(Cl)cc2)=C1C=O]

which is first reduced to the prochiral allylic alcohol of the formula (IV), which is then epoxidized to obtain either the racemic epoxy alcohol of the formula (V), or the enantiomers of the formulae (Va) or (Vb), if epoxidation of the prochiral allylic alcohol of the formula (IV) is performed in an enantioselective manner.

2-(4-Chlorobenzyl)-5,5-dimethylcyclopent-1-enecarbaldehyde of the formula (III) can be prepared in high yields by acid catalyzed isomerisation of the readily available 7-[1-(4-chlorophenyl)-meth-(E)-ylidene]-4,4-dimethyl-1-oxa-spiro[2.4]heptane of the formula (II) in yields higher than 90%.

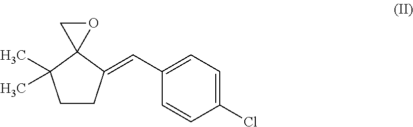 [SMILES: CC1(C)CC/C(=C\c2ccc(Cl)cc2)C12CO2]

As the compound of formula (II) is more readily available than the compound of formula (IX), the aldehyde of formula (III) is a much better starting material for the preparation of the epoxy alcohol of formulae (V), (Va) and (Vb), respectively, than the compound of formula (IX). In fact, in the process of EP 488396 compound (IX) is converted into the allylalcohole (IV) which is subsequently used as an intermediate for the preparation of cis-metconazole, as described herein above. However, the conversion of compound (IX) pursuant to the process of EP 488396 affords the allylalcohole (IV) in moderate yields at best, whereas the total yield of the 2-step conversion of compound (II) via the aldehyde (III) to the allylalcohole (IV) according to the process of the invention is in a much higher range of 80 to 90%.

Therefore, the present invention relates in a further aspect to a process for preparing the compounds of formulae (Ia) or (Ib) or mixtures thereof, e.g. racemic mixtures, which method comprises the following steps (a) to (d):
(a) providing the aldehyde of the formula (III);
(b) reducing the aldehyde of the formula (III) to obtain the allylic alcohol of the formula (IV);
(c) epoxidizing the allylic alcohol of the formula (IV) to obtain the epoxy alcohol of formulae (Va) or (Vb), or a mixture thereof;
(d) reacting the epoxy alcohol of formulae (Va) or (Vb), or a mixture thereof, e.g. the racemic mixture, with alkali metal borohydride or earth alkaline borohydride, in particular sodium borohydride, and anhydrous aluminum(III) chloride.

This process is hereinafter also termed as “Process B”.

The present invention relates in a further aspect to the aldehyde of formula (III).

In Process B, the aldehyde of the formula (III) is provided in step (a). The aldehyde of the formula (III) can be provided by treating 7-[1-(4-chlorophenyl)-meth-(E)-ylidene]-4,4-dimethyl-1-oxa-spiro[2.4]heptane of the formula (II) with an acid. The acid catalyzes the rearrangement of the compound of formula (II) into the aldehyde of formula (III).

Suitable acids for the treatment of the compound of formula (II) include hydrochloric acid, sulphuric acid or phosphoric acid with preference given to hydrochloric acid, in particular aqueous hydrochloric acid having concentration of hydrogen chloride of at least 20% w/w. The amount of acid is normally at least 0.5 mol per mol of the compound of formula (II) but an equimolar amount or an excess may be beneficial. Preferably the amount of acid is 0.5 mol to 2.0 mol, in particular 0.8 mol to 1.5 mol per mol of the compound of formula (II).

The treatment of the compound of formula (II) with the acid may be preferably performed at temperatures which do not exceed 50° C., in particular from 0 to 30° C., especially from 10 to 15° C.

It has generally been found to be advantageous to perform the rearrangement of (II) into (III) in an organic solvent for the reaction of process A which is sufficiently inert against acids under the reaction conditions. Useful organic solvents here include ethers, for example, aliphatic C₃-C₁₀-ethers having 1, 2, 3, or 4 oxygen atoms, such as glyme, diglyme, triglyme, diethyl ether, dipropyl ether, methyl isobutyl ether, tert-butyl methyl ether and tert-butyl ethyl ether, alicyclic C₄-C₆-ethers, such as THF, tetrahydropyrane, 2-methyltetrahydrofurane, 3-methyltetrahydrofurane and 1,4-dioxane, N,N-dimethylamides of C₁-C₄-carboxylic acids, such as dimethylformamide and dimethyl acetamide, N—C₁-C₄-alkyllactames such as N-methyl pyrrolidone, dipolar aprotic solvents such as acetonitrile or dimethylsulfoxide, aliphatic, cycloaliphatic or aromatic hydrocarbons, such as hexanes, heptanes, cyclohexane, methylcyclohexane, ethylcyclohexane, benzene, toluene, the xylenes and mesitylene, or mixtures of these solvents with one another. Particularly preferred are the aforementioned ethers, the dipolar aprotic solvents, the amides, the lactames and mixtures thereof with aromatic hydrocarbons, as well as the aromatic hydrocarbons. In a particular embodiment, step (a) is performed in an aromatic hydrocarbon or hydrocarbon mixture, specifically in toluene.

The aldehyde of formula (III) may be purified prior to step (b), e.g. by crystallization, or may be used as such in the reaction of step (b).

Step (b) can be performed by analogy to standard methods of reducing allylic carbaldehydes, as described e.g. in E. Keinan, N. Greenspoon in S. Patai, Z. Rappoport, The Chemistry of Enons, pt. 2, Wiley, NY, 1989, pp. 923-1022.

In a preferred embodiment of the invention the reduction in step (b) is performed using an alkali metal borohydride or an earth alkaline borohydride as reducing agent, in particular an alkali metal borohydride, especially sodium borohydride.

In this embodiment of step (b), the borohydride, in particular the alkali metal borohydride, especially sodium borohydride, is preferably used in an amount of 0.3 to 1.0 mol, more preferably 0.4 to 0.6 mol, based in each case on 1 mol of the aldehyde (III).

In this embodiment, step (b) is usually performed in an organic solvent. Preferred organic solvents for the reaction of (III) with the borohydride include but are not limited to ethers, for example, aliphatic C₃-C₁₀-ethers having 1, 2, 3, or 4 oxygen atoms, such as glyme, diglyme, triglyme, diethyl ether, dipropyl ether, methyl isobutyl ether, tert-butyl methyl ether and tert-butyl ethyl ether, alicyclic C₄-C₆-ethers, such as THF, tetrahydropyrane, 2-methyltetrahydrofurane, 3-methyltetrahydrofurane and 1,4-dioxane, mixtures thereof with aromatic hydrocarbons, such as toluene, the xylenes and mesitylene, as well as alkanols such as methanol, ethanol, n-propanol and isopropanol and mixtures thereof with aromatic hydrocarbons.

If the reduction in step (b) is performed by using a borohydride, the reaction temperature will preferably be in the range from 0 to 50° C.

In another preferred embodiment of the invention the reduction of the aldehyde (III) in step (b) is performed as a Meerwein-Ponndorf-Verley reduction, i.e. by reacting the aldehyde of formula (III) with an aluminium alkanolate of a secondary C₃-C₆-alkanol, such as the aluminium isopropylate or aluminium isobutylate (see e.g. C. F. Graauw et al., Synthesis 1994, pp. 1007-1017 and the literature cited therein). Alternatively, instead of an aluminium alkanolate of a secondary alkanol, an aluminium alkanolate of a tertiary alkanol, such as aluminium tert-butylate, can be used in combination with a secondary C₃-C₆-alkanol.

The aluminium alkanolate may be used in stoichiometric amount. It is however preferred to use an aluminium alkanolate of a secondary C₃-C₆-alkanol or a tertiary C₄-C₆-alkanol in substoichiometric amount together with a stoichiometric amount or an excess of the secondary alkanol such as isopropanol or 2-butanol, e.g. in an amount from 1 to 50 mol-%, in particular from 5 to 20 mol-% of aluminium alkanolate, based on the aldehyde (III), together with an excess of secondary alkanol, e.g. 1 to 10 mol of secondary alkanol per 1 mol of the aldehyde (III).

The reaction of the aldehyde (III) with substoichiometric amounts of aluminium alkanolate and the stoichiometric amount or excess of the secondary alkanol may be catalyzed by an acid, e.g. trifluoroacetic acid. The acid may be used in an amount from 10 to 50 mol-% per mol of the aluminium alkanolate.

If the reduction in step (b) is performed as a Meerwein-Ponndorf-Verley reduction, the reaction temperature will preferably be in the range from 20 to 150° C. The C₃-C₆-ketone formed in the reaction may be distilled off during the reaction.

If the reduction in step (b) is performed as a Meerwein-Ponndorf-Verley reduction, the reaction is preferably carried in an inert organic solvent. Suitable solvents include in particular secondary C₃-C₆-alkanols and aromatic hydrocarbons, such as toluene, the xylenes and mesitylene, as well as mixtures thereof. Preference is given to solvents which are essentially anhydrous, i.e. have a water content of less than 1000 ppm and especially not more than 100 ppm.

The allylic alcohol of the formula (IV) obtained in step (b) after conventional work-up, may be further purified or used in step (c) as such.

In step (c) of process B the allylic alcohol of the formula (IV) is epoxidized. The epoxidation of the allylic alcohol may be achieved by standard reactions of epoxidizing allylic alcohols as described e.g. in EP 488396.

Epoxidation is usually achieved by treating the allylic alcohol of the formula IV with an organic or inorganic hydroperoxide. Suitable hydroperoxides include but are not limited to hydrogen peroxide, alkylhydroperoxides, in particular secondary or tertiary alkylhydroperoxides such as tert.-butylhydroperoxide, isoamylhydroperoxide, tert.-amylhydroperoxide, alkylarylhydroperoxides such as cumene hydroperoxide and peroxycarboxylic acids such as peracetic acid and substituted perbenzoic acids such as metachloroperbenzoic acid.

The reaction temperature of the epoxidiation will preferably be in the range from −40 to 90° C. The reaction time will generally be in the range from 1 to 8 h.

The epoxidation of step c) will be generally performed in an organic solvent. Suitable solvents are those, which are inert against inorganic or organic hydroperoxides and include but are not limited to aliphatic hydrocarbons such as dichloromethane, 1,2-dichloroethane, dipolar aprotic solvents such as acetonitrile, aliphatic, cycloaliphatic or aromatic hydrocarbons, such as hexanes, heptanes, octanes, cyclohexane, methylcyclohexane, ethylcyclohexane, benzene, toluene, the xylenes and mesitylene, or mixtures of these solvents with one another.

The allylic alcohol of the formula (IV) is prochiral. Thus, it is possible to induce enantioselectivity by using chiral auxiliaries. Therefore, in a preferred embodiment of the in invention relates to a process where the allylic alcohol of the formula (IV) is enantioselectively converted into either the epoxy alcohol of formula (Va) or into the epoxy alcohol of formula (Vb). Suitable methods of enantioselectively epoxidizing an allylic alcohol of the formula (IV) into either the epoxy alcohol of formula (Va) or into the epoxy alcohol of formula (Vb) have been described e.g. in EP 488396.

A particularly useful method of performing step c) is the so-called “Sharpless epoxidation”, i.e. the reaction of the allylic alcohol (IV) with a hydroperoxide, e.g. hydrogen peroxide or an organic hydroperoxide in the presence of a titanium(IV) tetrakisalkanotate, in particular a titanium(IV) tetrakis (secondary C₃-C₆-alkanolate), specifically titanium(IV) tetrakis(2-propanolate), and a chiral auxiliary, in particular a di-C₁-C₆-alkyl ester of a chiral aliphatic dihydroxy-dicaroboxylic acid, especially a di-C₁-C₆-alkyl ester of tartaric acid such as (+) or (−)-diethyl tartrate and (+) or (−)-diisopropyl tartrate (see e.g. T. Katsuki et al. J. Am. Chem. Soc. 102 (1980), 5974-5976, K. B. Sharpless et al. Pure Appl. Chem. 55 (1983) p. 589 and p. 1823 and D. Schinzer in Organic Synthesis Highlights (H. Waldmann ed.) VCH Weinheim 1995, pp. 3-9 and literature cited therein. Preferred hydroperoxides are secondary or tertiary alkyl hydroperoxides such as tert-butyl hydroperoxide, isoamyl hydroperoxide and tert-amyl hydroperoxide. Sharpless epoxidation of the allylic alcohol (IV) yields either the epoxyalcohol Va or Vb in very high yields with enantiomeric excess of generally >80% ee and frequently >90% ee.

It has surprisingly been found that the aforementioned high yields and high enantiomeric excesses can also be achieved if the Sharpless epoxidation of the allylic alcohol (IV) is carried out in a solvent selected from aromatic hydrocarbons, such as benzene, toluene, the xylenes, mesitylene, or a mixture of these solvents. Thus, according to a preferred embodiment of the invention the Sharpless epoxidation of the allylic alcohol (IV) to either the epoxyalcohol Va or Vb is carried out in a solvent that comprises or preferably consists of aromatic hydrocarbon, in particular toluene. This way it is possible to perform the reaction sequence starting from compound (II) via the aldehyde (III) and the allylic alcohol (IV) to the epoxyalcohol Va or Vb in the same solvent, namely a hydrocarbon such as preferably toluene, without the need to in-between isolate the intermediates (III) and (IV) by completely or partly removing the solvent and afterwards re-dissolve them again. In fact, it is possible to introduce the solution of the raw compounds (III) and (IV) obtained in steps a) and b), respectively, directly into the following step b) and c), respectively. This way the process of the invention can be conducted much more economically, as not only less solvent and energy is needed but also the time required for carrying out the process is substantially reduced. It is therefore another preferred embodiment of the invention to carry out steps a), b) and c) of the process of the invention in the same solvent without intermediate complete or partial removal of the solvent. The solvent used pursuant to this embodiment comprises or preferably consist of an aromatic hydrocarbon, which preferably is toluene.

Furthermore, it has surprisingly been found that the starting compounds of process A, namely the epoxyalcohols of the formulae (Va) or (Vb), as well as their mixtures, in particular their racemic mixtures, can be prepared, starting from the compound of the formula (III), which is first epoxidized to obtain 5-(4-chloro-benzyl)-2,2-dimethyl-6-oxa-bicyclo[3.1.0]hexane-1-carbaldehyde, i.e. the epoxyaldehyde compound the formula (VIa) or (VIb) or a mixture thereof, in particular the racemic mixture (=compound of the formula (VI)).

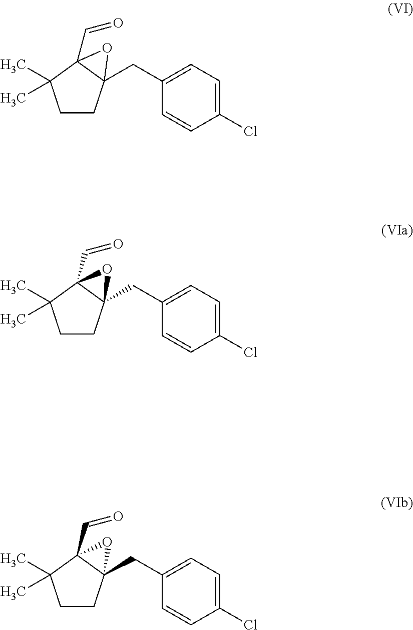 [SMILES: CC1(C)CCC2(Cc3ccc(Cl)cc3)OC12C=O ; CC1(C)CC[C@]2(Cc3ccc(Cl)cc3)O[C@]12C=O ; CC1(C)CC[C@@]2(Cc3ccc(Cl)cc3)O[C@@]12C=O]

The aldehyde group of (VIa) or (VIb) or a mixture thereof is subsequently reduced to obtain either the racemic epoxy alcohol of the formula (V), or the enantiomers of the formulae (Va) or (Vb), if epoxidation of the aldehyde of the formula (III) is performed in an enantioselective manner.

Therefore, the present invention relates in a further aspect to a process for preparing the compounds of formulae (Ia) or (Ib) or mixtures thereof, e.g. racemic mixtures, which process comprises the following steps (a), (b′), (c′) and (d):
(a) providing the aldehyde of the formula (III);
(b′) epoxidizing the aldehyde of the formula (III) to obtain an epoxylated aldehyde of the formula (VIa) or (VIb), or a mixture thereof, in particular a racemic mixture;
(c′) reducing the epoxy aldehyde of the formula (VIa) or (VIb), or a mixture thereof, with a reducing agent to obtain an epoxy alcohol of the formula (Va) or (Vb), or a mixture thereof;
(d) reacting the epoxy alcohol of formulae (Va) or (Vb), or a mixture thereof, e.g. the racemic mixture, with alkali metal borohydride or earth alkaline borohydride, in particular sodium borohydride, and anhydrous aluminum(III) chloride.

The process comprising steps (a), (b′), (c′) and (d) is hereinafter also referred to as Process C.

Epoxidation of the aldehyde (III) according to step (c′) of Process C can be achieved by analogy to the epoxidation of α,β-unsaturated ketones or aldehydes.

In a particular embodiment of the invention epoxidation of the aldehyde (III) is achieved by reacting the aldehyde (III) with hydrogen peroxide in the presence of a suitable base, e.g. by analogy to the methods described by Weitz and Scheffer in Ber. Dtsch. Chem. Ges. 54 (1921), p. 2327 or Paine et al. J. Org. Chem. 24 (1959), p. 54 and 26 (1961), p. 651.

Suitable bases for the reaction of step (b′) include alkali metal hydroxides, such as NaOH or KOH, alkali metal carbonates, such as sodium carbonate and potassium carbonate and alkali metal alkoxylates such sodium methanolate, sodium ethanolate, sodium isopropanolate, sodium tert.-butylate, potassium methanolate, potassium ethanolate, potassium isopropanolate and potassium tert.-butylate.

In step (b′), hydrogen peroxide is usually used in excess, i.e. the molar ratio of hydrogen peroxide to aldehyde (III) is typically ≧1:1, preferably from 1:1 to 2:1 and in particular from 1.01:1 to 1.5:1.

In step (b′), the base is usually used in sub-stoichiometric amounts, i.e. the molar ratio of base to aldehyde (III) is typically <1:1, preferably from 0.05:1 to 0.9:1 and in particular from 0.1:1 to 0.5:1.

Preferably, the reaction of step (b′) is performed in a solvent, which includes at least one C₁-C₄-alkanol as a main component, in particular in a solvent selected from C₁-C₄-alkanols and mixtures thereof such as methanol, ethanol, isopropanol, n-propanol and mixtures thereof.

The reaction of step (b′) is preferably performed at temperatures ranging from 0 to 30° C.

Reduction in step (c′) of the thus obtained epoxyaldehyde of formulae (VI), (VIa) and (VIb), respectively, can be achieved by analogy to the method described for step (b) of process B, thereby yielding the cis-diols of formulae (Ia) and (Ib) as well as mixtures thereof, in particular the racemic mixtures thereof.

The thus prepared cis-diols of formulae (Ia) and (Ib) as well as mixtures thereof, in particular the racemic mixtures thereof, are particularly useful for preparing cis-metconazole, in particular (−)-cis-metconazole and (+)-cis-metconazole.

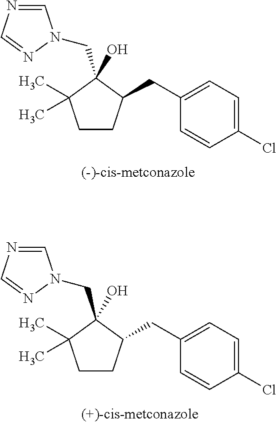 [SMILES: CC1(C)CC[C@H](Cc2ccc(Cl)cc2)[C@@]1(O)Cn1cncn1 ; CC1(C)CC[C@@H](Cc2ccc(Cl)cc2)[C@]1(O)Cn1cncn1]

The diols (Ia) and (Ib), as well as their mixtures can be converted into the corresponding sulfonic ester compounds (VIIa/VIIb) or into the spiro epoxides of formulae (IXa/IXb), e.g. by reacting the compound of formulae (Ia) or (Ib) or a mixtures thereof with a sulfonyl halide of the formula L-Hal, where Hal is chlorine or bromine in the presence of a base.

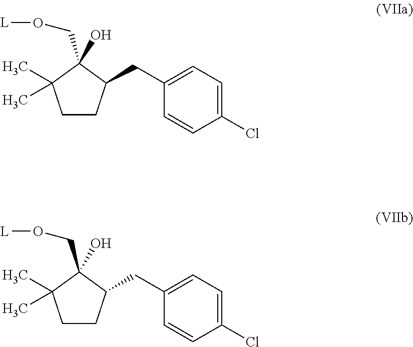

Herein, L is an optionally substituted alkylsulfonyl or arylsulfonyl group, preferably C₁-C₆-alkylsulfonyl such as methylsulfonyl, or phenylsulfonyl, which is unsubstituted or may carry a substituent selected from methyl, ethyl or chlorine, such as in 4-methylphenylsulfonyl.

Subsequent reaction of the compounds of formulae (VIIa) or (VIIb) or mixtures thereof with a triazolate salt of the formula (VIII)

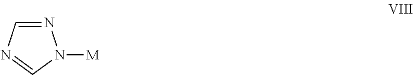

in which M is an alkali metal, preferably sodium or potassium, yields cis-methconazole, in particular (−)-cis-metconazole or (+)-cis-metconazole.

Likewise, subsequent reaction of the compounds of formulae (IXa) or (IXb) or mixtures thereof with a triazolate of the formula (VIII), yields cis-metconazole, in particular (−)cis-metconazole or (+)-cis-metconazole.

Therefore, the invention also relates to a process for the preparation of cis-metconazole, in particular to a process for preparing either (−)-cis-metconazole or (+)cis-metconazole, which comprises:
(i) providing the diol of the formulae (Ia) or (Ib), or a mixture thereof, e.g. a racemic mixture, by a process as described herein, in particular a process A, B or C;
(ii) converting the diol of the formula (Ia) or (Ib), or a mixture thereof, e.g. a racemic mixture, into a compound of the formula (VIIa) or (VIIb), or a mixture thereof,
(iii) reacting the a compound of the formula (VIIa) or (VIIb), or a mixture thereof, e.g.

a racemic mixture, with a triazolate salt of formula (VIII);

or, in the alternative:
(ii′) converting the diol of the formula (Ia) or (Ib), or a mixture thereof, into a compound of the formula (XIa) or (XIIb), or a mixture thereof, e.g. a racemic mixture,
(iii′) reacting the a compound of the formula (IXa) or (IXb), or a mixture thereof with a triazolate salt of formula (VIII).

According to the description herein, it should be clear that the use of a racemic mixture of cis-diols (Ia) and (Ib) will result in racemic cis-metconazole, while the use of cis-diol (Ia) will result in a selective synthesis of (−)-cis-metconazole and the use of cis-diol (Ib) will result in a selective synthesis of (+)-cis-metconazole.

Generally, an approximately stoichiometric amount of a base is used in step (ii), based on the amount of compound (Ia) and (Ib). The base can, however, also be used in a superstoichiometric amount. In general, the base is used in an amount of from 0.5 to 10 mol and especially in the amount of from 0.9 to 5 mol per mol (Ia/Ib). Preference is given to working with an amount of from 1 to 3 mol per mol (Ia/IIb).

Suitable bases for the reaction of step (ii) are organic and inorganic bases.

Suitable inorganic bases are, for example, alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate. Preference is given to an aqueous NaOH solution or an aqueous KOH solution.

The organic base advantageously is an amine base, i.e. a base wherein the site of basicity is a nitrogen atom. Preferably, the amine base is a tertiary alkyl-, alkenyl-, or alkinylamine or an arylamine or a heterocyclic aromatic amine. Preference is given to triethylamine, dimethylcyclohexylamine, diisopropylethylamine and tri-n-butylamine, N-methyl pyrrolidine, N-methyl piperidine, N-methyl morpholine, N,N′-dimethyl piperazine, DABCO (1,4-diazabicyclo[2.2.2]octane), DBU (1,8-diazabicyclo[5.4.0]undec-7-ene), DBN (1,5-diazabicyclo[4.3.0]non-5-ene), DBU (1,8-diazabicyclo[5.4.0]undec-7-ene) and DBN (1,5-diazabicyclo[4.3.0]non-5-ene), pyridine, 2-picoline, 3-picoline, 4-picoline, 5-ethyl-2-methylpyridine, 2-ethylpyridine, 2,3-lutidine, 2,4-lutidine, 2,5-lutidine, 2,6-lutidine, 3,4-lutidine, 3,5-lutidine, 2,4,6-collidine, 2,3,5-collidine.

Typically, the conversion of (VIIa)/(VIIb) or (IXa/IXb) into cis-metconazole is effected in an inert dipolar aprotic organic solvent. Examples of such solvents are nitriles such as acetonitrile and propionitrile, dimethylformamide, dimethyl-acetamide, N-methylpyrrolidone, dimethylsulfoxide mixtures thereof. The preference is given to dimethylformamide and N-methylpyrrolidone.

The starting oxirane of the formula (II) can be obtained from adipinic acid dimethylester according to EP-A 751111 via the ketone (XV)

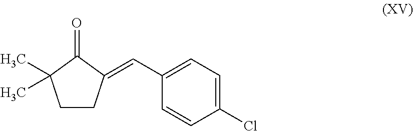 [SMILES: CC1(C)CC/C(=C\c2ccc(Cl)cc2)C1=O]

and subsequent conversion with dimethylsulfoniummethylid according to EP-A 467792. Dimethylsulfoniummethylid is obtainable from trimethylsulfonium salts in the presence of a base according to E. J. Corey, M. Chaykovsky, JACS 87, 1965, p. 1313ff.

Hereinafter, the following abbreviations are used:

aq.=aqueous

wt-%=% by weight

% ee=enantiomeric excess

DME=Dimethoxyethane (glyme)

DMF=Dimethyl formamide

EtOH=Ethanol

MCH=methylcyclohexane

Ti(OiPr)₄=Titanium(IV) tetrakis(2-propanolat)

(+)-DET=(+)-diethyltartrate

The magnetic nuclear resonance spectral properties (NMR) refer to the chemical shifts (6) expressed in parts per million (ppm). The relative area of the shifts in the ¹H NMR spectrum corresponds to the number of hydrogen atoms for a particular functional type in the molecule. The nature of the shift, as regards multiplicity, is indicated as singlet (s), broad singlet (s. br.), doublet (d), broad doublet (d br.), triplet (t), broad triplet (t br.), quartet (q), quintet (quint.) and multiplet (m)

### EXAMPLE 1

### 2-(4-Chloro-benzyl)-5,5-dimethyl-cyclopent-1-enecarbaldehyde (III)

371 g of a 26.7 wt-% solution of 7-[1-(4-chlorophenyl)-meth-(E)-ylidene]-4,4-dimethyl-1-oxa-spiro[2.4]heptane (II) in toluene were added at 10-15° C. to a stirred pre-cooled mixture of 37.5 g aq. hydrochloric acid (36 wt-%) and 129 g toluene within 1 h. After 3 h at 15° C. the toluene solution was separated from the aqueous phase. The toluene solution contained 17.25 wt-% of the product, determined by quantitative HPLC-assay, corresponding to a yield of 90.9%.

The compound of formula (III) can be isolated as a solid after evaporation of the toluene solution and crystallization from isopropanol at 0° C.

Melting point: 57-59° C.

CI-MS: m/e=249 (M+H)

Elemental analysis: C, 72.4%; H, 6.9%; O, 6.3%; Cl 14.4%

¹H-NMR (500 MHz, CDCl₃): δ/ppm=1.25 (s, 6H), 1.66 (t, 2H), 2.33 (t, 2H), 3.83 (s, 2H), 7.10 (d, 2H)), 7.30 (d, 2H), 10.13 (s, 1H)

¹³C-NMR (125 MHz, CDCl₃): δ/ppm=26.83 (q, 2C), 33.98 (t), 34.54 (t), 38.87 (t), 46.03 (s), 128.96 (d, 2C), 129.9 (d, 2C), 132.54 (s), 136.22 (s), 145.07 (s), 161.98 (s), 188.51 (s)

IR: ν/cm⁻¹=2734, 1666. 1617, 1491, 1086, 842, 799

### EXAMPLE 2

### [2-(4-Chloro-benzyl)-5,5-dimethyl-cyclopent-1-enyl]-methanol (IV

20 g of 2-(4-chloro-benzyl)-5,5-dimethyl-cyclopent-1-enecarbaldehyde (III), purity 97.1 wt-%, were added at 25° C. to a solution of 1.2 g sodium borohydride in 25 g methanol. The reaction mixtures warmed to 50° C. and gas evolution occurred during addition of the borohydride. Additional 0.4 g of sodium borohydride were added at 25° C. to achieve complete conversion. After 2 h at 25° C. the reaction mixture was added to toluene and water. After phase separation the toluene phase was washed two times with water. After evaporation of solvents the product was obtained as 19.8 g of a brown oil with a purity of 90.1 wt-%, determined by quantitative HPLC-assay, corresponding to a yield of 91.2%.

¹H-NMR (500 MHz, CDCl₃): δ/ppm=1.12 (s, 6H), 1.18 (s broad, OH) 1.63 (t, 2H), 2.14 (t, 2H), 3.44 (s, 2H), 4.23 (s, 2H), 7.08 (d, 2H)), 7.25 (d, 2H)

¹³C-NMR (125 MHz, CDCl₃): δ/ppm=27.50 (q, 2C), 32.59 (t), 34.43 (t), 39.24 (t), 46.59 (s), 56.31 (t), 128.49 (d, 2C), 129.78 (d, 2C), 131.70 (s), 138.26 (s), 138.80 (s), 143.62 (s)

### EXAMPLE 3

### [2-(4-Chloro-benzyl)-5,5-dimethyl-cyclopent-1-enyl]-methanol (IV

839 g of a 26.7 wt-% solution of 7-[1-(4-chloro-phenyl)-meth-(E)-ylidene]-4,4-dimethyl-1-oxa-spiro[2.4]heptane (II) in toluene were added within 1.5 h at 10-15° C. to a stirred pre-cooled mixture of 88.7 g of aq. hydrochloric acid (37 wt-%) and 250 g toluene. After 3 h at 15° C. the toluene solution was separated from the aqueous phase and washed with 211 g of a saturated aqueous solution of NaHCO₃. The toluene solution was added at 25° C. to a mixture of 250 g methanol and 135 g of a 12 wt-% solution of sodium borohydride in 14 molar aqueous solution of sodium hydroxide. After 3 h at 25° C. 250 g water were added to the reaction mixture. The aqueous phase was discarded. The toluene solution was washed twice with 200 g water and then evaporated to yield 319 g of an oily residue containing 64.0 wt-% of the product as determined by quantitative HPLC-assay, corresponding to a yield of 85%

### EXAMPLE 4

### [2-(4-Chloro-benzyl)-5,5-dimethyl-cyclopent-1-enyl]-methanol (IV

92.7 g a 26.7 wt-% solution of 7-[1-(4-chloro-phenyl)-meth-(E)-ylidene]-4,4-dimethyl-1-oxa-spiro[2.4]heptane (II) in toluene were added within 1.5 h at 10-15° C. to a stirred pre-cooled mixture of 9.4 g cf aq. hydrochloric acid (37 wt-%) and 32.3 g toluene. After 3 h at 15° C. the toluene solution was separated from the aqueous phase and washed with 25 g of a saturated aqueous solution of NaHCO₃. The toluene solution was azeotropically dried by distilling off about 33 g of toluene. Then 21 g of aluminium isopropoxide were added and the solution was kept at 50° C. for 3 h and then stirred overnight at 25° C. Then 200 g of dilute aqueous sulfuric acid (10 wt-%) were added to the reaction mixture. After phase separation the aqueous phase was extracted with 50 g of toluene.

The combined toluene solution was as azeotropically dried by distillation. 102.4 g of a clear yellow-red solution were obtained containing 18.8 wt-% of the product as determined by quantitative GC-assay, corresponding to a yield of 77%.

### EXAMPLE 4A

### [2-(4-Chloro-benzyl)-5,5-dimethyl-cyclopent-1-enyl]-methanol (IV

428 g of a 25.0 wt-% solution of 7-[1-(4-chloro-phenyl)-meth-(E)-ylidene]-4,4-dimethyl-1-oxa-spiro[2.4]heptane (II) in toluene were added within 1 h at 10-15° C. to a stirred pre-cooled mixture of 41.2 g conc. hydrochloric acid (37 wt-%) and 142 g toluene. After 2 h at 15° C. the reaction mixture was warmed to 25° C. and the toluene solution was separated from the aqueous phase. The toluene solution was azeotropically dried by distillation of ca. 89 g toluene at 50° C./90 mbar. The solution was added at 25° C. to a pre-charged mixture of 200 g isopropanol, 9.2 g aluminium isopropoxide, 17.8 g toluene and 2.5 g trifluoroacetic acid. After stirring at 25° C. for 20 h the reaction mixture was set under reduced pressure (200 mbar) for 30 min. Then 200 g of dilute sulfuric acid (10 wt-%) were added. After phase separation the toluene solution was washed twice with 200 g water. Finally the toluene solution was concentrated and azeotropically dried by stripping off 185 g distillate at 50° C./700 mbar. 382.7 g of a clear yellow-red solution were obtained containing 24.7 wt-% of the product as determined by quantitative GC-assay, corresponding to a yield of 86%.

### EXAMPLE 5

### racemic 5-(4-chloro-benzyl)-2,2-dimethyl-6-oxa-bicyclo[3.1.0]hex-1-yl]-methanol (V)

311.5 g of the oil prepared in example 3 were charged into a 1.6 L vessel with jacket cooling together with 15 g of toluene (flushing volume). 1.3 g vanadyl acetoacetonate were added to the starting material. Then, 474 g of a solution of tert-butylhydroperoxide in toluene prepared by extraction of 123 g of aqueous tert-butylhydroperoxide (70 wt-%) with 414 g toluene were added rapidly in one portion (jacket temp. 10° C.), whereby the mixture warmed to 85° C. After cooling to 25° C. the reaction mixture was stirred for 1 h. Then a 30 wt-% aqueous solution of FeSO₄×7H₂O was added until the test for residual peroxide remained negative. The toluene phase was treated with 100 g of 2 N aqueous hydrochloric acid, subsequently washed with 250 g water and then evaporated at 50° C./1 mbar. The residue was dissolved in 627 g of methylcyclohexane at 50° C. and then cooled for crystallization with a rate of 6K/h to 0° C. The crystallized product was filtered and washed two times with methylcyclohexane (43 g and 127 g) and dried in a vacuum dryer at 25° C. 174 g of product with a purity of 96.3 wt-%, determined by quantitative HPLC-assay, were obtained, corresponding to a yield of 79%.

Melting point: 91° C.

¹H-NMR (500 MHz, CDCl₃): δ/ppm=0.94 (s, 3H), 1.12 (s, 3H), 1.1-1.3 (m, 2H), 1.5-1.75 (m, 2H), 2.7 (s broad, OH), 3.02 (d, 1H), 3.08 (d, 1H), 3.93 (d, 1H), 4.13 (d, 1H), 7.18 (d, 2H)), 7.28 (d, 2H)

¹³C-NMR (125 MHz, CDCl₃): δ/ppm=23.02 (q), 23.47 (q), 28.00 (t), 34.61 (t), 36.10 (t), 40.68 (s), 58.04 (t), 72.46 (s), 73.99 (s), 128.50 (d, 2C), 130.48 (d, 2C), 132.24 (s), 136.18 (s)

IR: ν/cm⁻¹=3415, 2965, 2889, 1493, 1365, 1088, 1053, 909, 845, 804, 737

### EXAMPLE 6

### racemic 5-(4-chloro-benzyl)-2,2-dimethyl-6-oxa-bicyclo[3.1.0]hexane-1-carbaldehyde (VI)

10 g of 2-(4-chloro-benzyl)-5,5-dimethyl-cyclopent-1-enecarbaldehyde (III), 98 wt-% and 2.1 g of a 30 wt-% solution of sodium methylate in methanol were dissolved in 45 g methanol. The mixture was cooled to 15° C. and then 6.7 g of hydrogen peroxide in water (30 wt-%) were added within 10 min, whereby the mixture warmed to 30° C. After 1 h at 25° C. the reaction mixture was added to a mixture of 50 g water and 80 g toluene. The toluene phase was washed with 50 g water and evaporated at 50° C./1 mbar. Thereby, 8.3 g of the product were obtained as a wax with a purity of 93.4 wt-%, determined by quantitative HPLC-assay, corresponding to a yield of 74.3%.

EI-MS: m/e=264 (Mt)

¹H-NMR (500 MHz, CDCl₃): δ/ppm=1.09-1.35 (m, 2H), 1.13 (s, 3H), 1.21 (s, 3H) 1.68-1.84 (m, 2H), 3.04 (d, 1H), 3.13 (d, 1H), 7.14 (d, 2H)), 7.28 (d, 2H), 9.55 (s, 1H)

¹³C-NMR (125 MHz, CDCl₃): δ/ppm=22.58 (q, 1C), 23.13 (q, 1C), 28.59 (t), 34.84 (t), 36.14 (t), 40.94, (s), 75.06 (s), 75.25 (s), 128.80 (d, 2C), 130.54 (d, 2C), 132.87 (s), 135.07 (s), 198.11 (s)

IR: ν/cm⁻¹=2960, 1716, 1492, 1091, 1016, 844, 823

### EXAMPLE 7

### racemic [5-(4-chloro-benzyl)-2,2-dimethyl-6-oxa-bicyclo[3.1.0]hex-1-yl]-methanol (V)

90 g of a 26.0 wt-% solution of 7-[1-(4-chloro-phenyl)-meth-(E)-ylidene]-4,4-dimethyl-1-oxa-spiro[2.4]heptane (II) in toluene were added within 1 h at 15-20° C. with stirring to 9.2 g of pre-cooled conc. hydrochloric acid (36-wt-%). After 2 h at 25° C. the toluene solution was separated from the aqueous phase. The toluene solution was washed with 30 ml of a saturated aqueous solution of NaHCO₃ and then evaporated at 50° C./1 mbar. The residue was added to a mixture of 144 g methanol and 4.4 g sodium methylate solution in methanol (30 wt-%) at 10° C. Then 13.6 g of hydrogen peroxide in water (30 wt-%) were added in 1 h at 10-25° C. After 1 h at 25° C. 15 g of sodium borohydride (12 wt-%) in 14 molar aqueous solution of sodium hydroxide were added in 20 min at 20-25° C. After 2 h at 25° C. a test for residual peroxide remained negative.

The mixture was then evaporated at 50° C./100 mbar. 50 g water and 100 g toluene were added to the residue. Residual borohydride was destroyed by addition of 70 g of 2 molar hydrochloric acid. Thereafter, the toluene solution was evaporated at 50° C./1 mbar. The product was obtained as 24.3 g of a yellow oil with a chemical purity of 77.3 wt-% determined by quantitative HPLC, corresponding to a yield of 74.9%.

### EXAMPLE 8

### racemic cis-5-(4-Chloro-benzyl)-1-hydroxymethyl-2,2-dimethylcyclopentanol (racemic mixture of Ia/Ib)

2.0 g solid sodium borohydride were added to 65 g of DME at 0° C. After 30 min of stirring at 0° C., when most of the solid had been dissolved, 4.7 g anhydrous aluminum(III)chloride were added. Stirring at 0° C. was continued for further 30 min resulting in an almost clear solution. To this clear solution a solution of 10 g 94.9 wt-% racemic 5-(4-Chloro-benzyl)-2,2-dimethyl-6-oxa-bicyclo[3.1.0]hex-1-yl]-methanol (V) prepared according to example 5, in 30 g DME was added within 7 min with cooling at 0° C. Gas evolution occurred during the addition. Then the reaction mixture was stirred for 0.5 h at 0° C., followed by stirring for 1 h at 25° C. and for further 1 h at 50° C. Then most of the DME as evaporated at 50° C./100 8 mbar. The thus obtained residue was treated at 50° C. with 90 g toluene and 90 g of a 2 molar aqueous solution of sodium hydroxide. The toluene solution was partly concentrated to 65 g of a slightly yellow clear solution containing 13.6 wt-% of the product determined by quantitative HPLC, corresponding to a yield of 91.9%.

An analytical sample was prepared by crystallization from toluene.

Melting point: 102° C.

¹H-NMR (400 MHz, CD₃CN): δ/ppm=0.89 (s, 3H), 1.02 (s, 3H), 1.18-1.38 (m, 2H), 1.45-1.58 (m, 1H), 1.62-1.73 (m, 1H), 2.17-2.27 (m, 1H), 2.41-2.49 (dd, 1H), 2.8 (s, OH) 2.86-2.95 (dd, 1H), 2.94 (s, OH), 3.60-3.73 (m, 2H), 7.18 (d, 2H), 7.24 (d, 2H)

¹³C-NMR (125 MHz, CD₃CN): δ/ppm=23.76 (q), 25.75 (q), 27.88 (t), 36.86 (t), 39.16 (t), 46.42 (s), 47.14 (d), 65.69 (t), 129.04 (d, 2C), 131.57 (d, 2C), 131.61 (s), 142.44 (s)

### EXAMPLE 9

### racemic cis-5-(4-Chlorobenzyl)-1-hydroxymethyl-2,2-dimethylcyclopentanol (racemic mixture of Ia/Ib)

1.7 g solid sodium borohydride were added to 65 g 1.2 DME at 0° C. After 30 min of stirring at 0° C., when most of the solid had been dissolved, 4.0 g of anhydrous aluminum(III)chloride were added. Stirring at 0° C. was continued for further 30 min resulting in an almost clear solution. To this clear solution a solution of 10 g 94.9 wt-% [5-(4-Chloro-benzyl)-2,2-dimethyl-6-oxa-bicyclo[3.1.0]hex-1-yl]-methanol (V), prepared according to example 5, in 30 g DME under cooling in 7 min at 0-4° C. Gas evolution occurred during the addition. Then the reaction mixture was stirred for 6 h at 0° C. and 1 h at 25° C. Then most of the DME was evaporated at 50° C./100-8 mbar. The residue was treated with 90 g toluene and 90 g of 2 molar aqueous sodium hydroxide solution at 50° C. and again with 22 g of 2 molar aqueous sodium hydroxide solution at 50° C. The 101 g toluene solution obtained after phase separation contains 7.9 wt-% of the product determined by quantitative HPLC, corresponding to a yield of 82.0%.

### EXAMPLE 10

### 3SR,7RS)-7-(4-Chloro-benzyl)-4,4-dimethyl-1-oxa-spiro[2.4]heptane (racemic mixture of IXa/IXb)

108 g of a solution, prepared according to example 9, were concentrated and azeotropically dried partial evaporation of toluene/water to yield 61.6 g of a solution containing 13.6 wt-% of racemic 5-(4-chloro-benzyl)-1-hydroxymethyl-2,2-dimethyl-cyclopentanol. To this solution 6.1 g of dimethylcyclohexylamine were added. Then the reaction mixture was cooled to 5° C. and 10 g of a 50 wt-% solution of methanesulfonylchloride in toluene were added within 1 h at 5-10° C. After stirring for additional 0.5 h at 5° C., 56.5 g of a 10 wt-% aqueous solution of sodium hydroxide were added whereby the reaction mixtures warmed to 25° C. The resulting mixture was stirred for 4 h at 25° C. Then the aqueous phase was separated and the organic phase was extracted with 30 g of 2 molar hydrochloric acid at pH1 and then washed with 21 g of saturated aqueous solution of sodium bicarbonate and 69 g water. Then the solvent was evaporated at 50° C./1 mbar. 9 g of the product were obtained as a yellow oil with a purity of 78.0 wt-%, determined by quantitative GC-analysis, corresponding to a yield of 89.7%.

### EXAMPLE 11

### Synthesis of racemic 5-(4-chloro-benzyl)-2,2-dimethyl-1-[1,2,4]triazol-1-ylmethyl-cyclopentanol [cis-metconazole]

A suspension of 18 g sodium triazolide in 170 g DMF was heated to 90° C. At this temperature 47.3 g of 78.3 wt-% racemic (3SR,7RS)-7-(4-chloro-benzyl)-4,4-dimethyl-1-oxa-spiro[2.4]heptane (racemic mixture of IXa/IXb), prepared according to example 10, were added, followed by stirring of the mixture for 6 h at 90° C. Then 129 g of the DMF were distilled off at 50° C./3 mbar. To the resulting residue 87 g water and 320 g of MCH were added and the phases were separated at 65° C. The aqueous phase was extracted with 100 g fresh MCH at 65° C. The combined MCH solutions were washed three times with 80 g water. Thereafter the MCH solution was concentrated to 309 g at 80° C./500-400 mbar. For crystallization the solution was cooled from 80° C. to 0° C. with a rate of 6° K/h. After stirring overnight the product was isolated by filtration. The filter cake was washed twice with 50 g of ice-cold MCH and then dried in a vacuum dryer at 50° C./8 mbar. Thus 36.4 g of cis-metconazole were obtained, having a purity of 97.1%, determined by quantitative GC-analysis, corresponding to a yield of 74.5%.

Melting point: 112° C.

¹H-NMR (500 MHz, CDCl₃): δ/ppm=0.62 (s, 3H), 1.04 (s, 3H), 1.28-1.48 (m, 2H), 1.64-1.82 (m, 2H), 2.25-2.52, (m, 3H), 3.87-3.94 (s, broad OH), 4.19 (d, 1H) 4.28 (d, 1H), 7.10 (d, 2H), 7.20 (d, 2H), 7.98 (s, 1H), 8.21 (s, 1H)

¹³C-NMR (125 MHz, CDCl₃): δ/ppm=21.94 (q), 25.08 (q), 27.20 (t), 35.83 (t), 38.07 (t), 46.29 (s), 46.86 (d), 53.92 (t), 82.37 (s), 128.36 (d, 2C), 130.19 (d, 2C), 131.46 (s), 139.71 (s), 144.30 (d), 151.37 (d)

### EXAMPLE 12

### [(1S,5R)-5-(4-Chloro-benzyl)-2,2-dimethyl-6-oxa-bicyclo[3.1.0]hex-1-yl]-methanol (Va)

3 g of Ti(OiPr)₄ were added at −20° C. to a stirred suspension of 10.5 g powdered molecular sieves (4 A) in 260 g toluene. Then 3 g of (+)-DET and 26 g of ([2-(4-chloro-benzyl)-5,5-dimethyl-cyclopent-1-enyl]-methanol (IV), prepared according to example 3 and having a purity of 86.5 wt-%, were added and the mixtures was stirred for 30 min at −20° C. Then, 32 g of a anhydrous solution of tert-butylhydroperoxide in nonane (59 wt-%) were added at the same temperature. The obtained mixture was stirred for 2 h at −20 to −30° C. and then warmed to 25° C. After 1 h at 25° C., the mixture was filtered though a celite pad to remove the molecular sieves. The filter pad was washed several times with toluene. The collected toluene filtrates were added slowly at 0° C. to 300 g of an aqueous solution containing 30 wt-% FeSO₄×7H₂O and 7.8 wt-% citric acid. The mixture was stirred for 30 min at 0-10° C. and then again filtered through a celite pad to improve phase separation. The toluene solution was treated with 52 g 2 N hydrochloric acid, washed with 109 g water and then evaporated at 50° C./2 mbar. Thereby 27.3 g of the product were obtained as a yellow oil with a chemical purity of 82.3 wt-% determined by quantitative HPLC. The product had 96% ee as determined by HPLC using a Chiralpak AD-RH 5 μm+4.6 mm column from Daicel. Based on the chemical purity the yield was 93.9%.

[α]_{D}²⁰: +16.5° (c=1.48, EtOH)

### EXAMPLE 13

### 1S,5R)-5-(4-Chloro-benzyl)-1-hydroxymethyl-2,2-dimethyl-cyclopentanol (Ia

5.4 g of solid sodium borohydride were added to 175 g DME at 0° C. and the mixtures was stirred for 30 min at 0° C., until most of the solid had been dissolved. Then. 12.7 g of solid aluminum chloride were added and after further 30 min of stirring at 0° C. an almost clear solution resulted. To this solution a solution of 26.8 g [(1S,5R)-5-(4-chloro-benzyl)-2,2-dimethyl-6-oxa-bicyclo[3.1.0]hex-1-yl]-methanol (Va) having a purity of 82.3 wt-% and prepared according to example 12, in 66 g DME was added with cooling to 0° C. within 8 min. Gas evolution occurred during the addition. Then the reaction mixture was stirred for 0.5 h at 0° C., 1 h at 25° C. and 1 h at 50° C. Then most of the DME was evaporated at 50° C. at reduced pressure from 100 to 8 mbar. The thus obtained residue was treated at 50° C. for 1 h with 241 g toluene and 229 g of a 2 molar aqueous solution of sodium hydroxide. The toluene solution was partly concentrated to yield 139 g of a slightly yellow clear solution containing 14.0 wt-% of the product as determined by quantitative HPLC, corresponding to a yield of 87.4%. A sample of the solution was evaporated. From the residue the enantiomeric excess of the product was determined to be 96% ee by HPLC using a Chiralpak AD-RH 5 μm+4.6 mm column from Daicel.

[α]_{D}²⁰: +33.2° (c=0.76, EtOH)

### EXAMPLE 14

### 3S,7R)-7-(4-Chloro-benzyl)-4,4-dimethyl-1-oxa-spiro[2.4]heptane (IXa

To 134.7 g of the solution, prepared in example 13, were added 13.9 g of dimethylcyclohexylamine. After cooling to 5° C. 22.8 g of a 50 wt-% solution of methanesulfonylchloride in toluene were added within 1 h at 5-10° C. After stirring for additional 0.5 h at 5° C. 127 g of a 10 wt-% aqueous solution of sodium hydroxide were added whereby the resulting mixture warmed to 25° C. The mixture was stirred for 4 h at 25° C. Then the aqueous phase was separated off and the toluene solution with extracted with 56 g of a 2 M hydrochloric acid at pH 1 followed by washing with 37 g of saturated aqueous sodium bicarbonate and 40 g water. Then the solvent was evaporated at 50° C./1 mbar and 24.6 g of the product were obtained as a yellow oil with a purity of 64.1 wt-%, determined by quantitative GC-analysis, corresponding to a yield of 89.7%. From a sample of the residue the optical rotation was measured:

[α]_{D}²⁰: +30.0° (c=0.77, EtOAc)

### EXAMPLE 15

### 1S,5R)-5-(4-Chloro-benzyl)-2,2-dimethyl-1-[1,2,4]triazol-1-ylmethyl-cyclopentanol; (−)-cis-metconazole

A suspension of 7.5 g sodium triazolide in 70 g DMF was heated to 90° C. At this temperature 23 g of (3S,7R)-7-(4-chloro-benzyl)-4,4-dimethyl-1-oxa-spiro[2.4]heptane (IXa) having a purity of 64.1 wt-% and prepared according to example 14, were added and the resulting mixture was stirred for 6 h at 90° C. Then 55 g of DMF were distilled off at 50° C./3 mbar. To the resulting residue 37 g water and 130 g MCH were added and the phases were separated at 65° C. The aqueous phase was extracted with 50 g of fresh MCH at 65° C. The combined MCH solutions were washed twice with 30 g water at 80° C. Thereafter the MCH solution was concentrated to 124 g at 80° C./500-400 mbar. For crystallization the solution was cooled from 80° C. to 0° C. with a rate of 6° K/h. After stirring overnight 12.2 g of crystallized product were collected and re-dissolved in 98 g of fresh MCH at 98° C. and re-crystallized by cooling to 0° C. with a rate of 9° K/h. After filtration the filter cake was washed twice with 5 g of ice cold MCH and then dried in a vacuum dryer at 50° C./8 mbar. Thus 10 g of (−)-cis-metconazole with a purity of 95.1%, determined by quantitative GC-analysis, were obtained, which corresponds to a yield of 50.6%. The enantiomeric excess of the product was determined to be 99.2% ee by HPLC using a Chiracel OD-RH 150×4.6 mm column from Daicel.

Melting point: 134° C.

[α]_{D}²⁰: −26.2° (c=2,48, EtOH)

¹H-NMR (500 MHz, CDCl₃): δ/ppm=0.61 (s, 3H), 1.04 (s, 3H), 1.28-1.50 (m, 2H), 1.65-1.82 (m, 2H), 2.25-2.52, (m, 3H), 3.6-3.78 (s, broad OH), 4.19 (d, 1H) 4.28 (d, 1H), 7.10 (d, 2H), 7.20 (d, 2H), 8.0 (s, 1H), 8.18 (s, 1H)

13C-NMR (125 MHz, CDCl₃): δ/ppm=21.93 (q), 25.09 (q), 27.21 (t), 35.82 (t), 38.08 (t), 46.29 (s), 46.87 (d), 53.87 (t), 82.37 (s), 128.53 (d, 2C), 130.15 (d, 2C), 131.46 (s), 139.67 (s), 144.27 (d), 151.43 (d)

1-17. (canceled)

18. A process for preparing a diol of the formulae (Ia) or (Ib), or a mixture thereof, 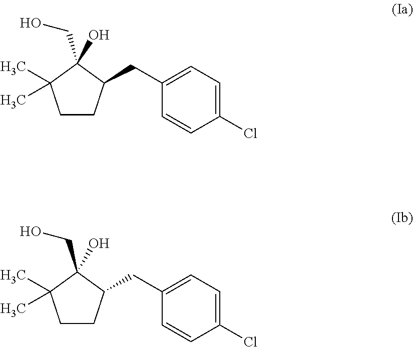 [SMILES: CC1(C)CC[C@H](Cc2ccc(Cl)cc2)[C@@]1(O)CO ; CC1(C)CC[C@@H](Cc2ccc(Cl)cc2)[C@]1(O)CO]
comprising reacting an epoxy alcohol of formulae (Va) or (Vb), or a mixture thereof, 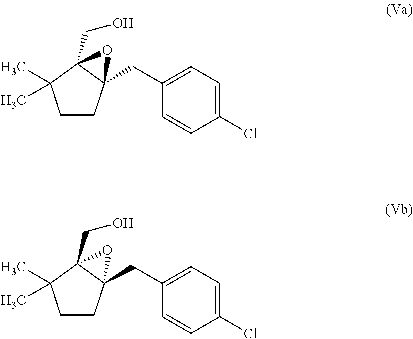 [SMILES: CC1(C)CC[C@]2(Cc3ccc(Cl)cc3)O[C@]12CO ; CC1(C)CC[C@@]2(Cc3ccc(Cl)cc3)O[C@@]12CO]
with alkali metal borohydride selected from sodium borohydride and potassium borohydride, and anhydrous aluminum(III) chloride.

19. The process of claim 18, wherein the reaction with sodium borohydride and aluminum(III) chloride is carried out in an organic solvent selected from ethers.

20. The process of claim 19, wherein the organic solvent is selected from tetrahydrofuran, dioxane, glyme, diglyme, triglyme and mixtures thereof.

21. The process of claim 18, wherein the borohydride is used in an amount of 0.8 to 2.0 mol and the aluminum(III) chloride is used in an amount of 0.4 to 1.1 mol, based in each case on 1 mol of the epoxy alcohol of formula (Va) or (Vb), or a mixture thereof.

22. The process of claim 21 wherein the borohydride is used in an amount of 1.2 to 1.5 mol, based on 1 mol of the epoxy alcohol of formula (Va) or (Vb), or a mixture thereof.

23. The process of claim 18, which further comprises:
(a) providing an aldehyde of the formula III; 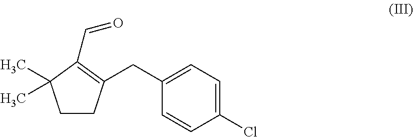 [SMILES: CC1(C)CCC(Cc2ccc(Cl)cc2)=C1C=O]
(b) reducing the aldehyde of the formula (III) to obtain the allylic alcohol of the formula IV; 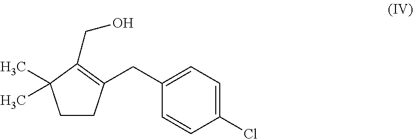 [SMILES: CC1(C)CCC(Cc2ccc(Cl)cc2)=C1CO]
(c) epoxidizing the allylic alcohol of the formula (IV) to obtain the epoxy alcohol of formula (Va) or (Vb), or a mixture thereof.

24. The process of claim 23, wherein the reduction in step (b) is performed using an alkali metal borohydride as reducing agent.

25. The process of claim 23, wherein the reduction in step (b) is a Meerwein-Ponndorf-Verley reduction.

26. The process of claim 23, wherein in step (c) the allylic alcohol of the formula (IV) is enantioselectively converted into the epoxy alcohol of formula (Va) or into the epoxy alcohol of formula (Vb).

27. The process of claim 18, which comprises:
(a) providing an aldehyde of the formula III;
(b′) epoxidizing the aldehyde of the formula (III) to obtain an epoxylated aldehyde of the formula (VIa) or (VIb), or a mixture thereof; 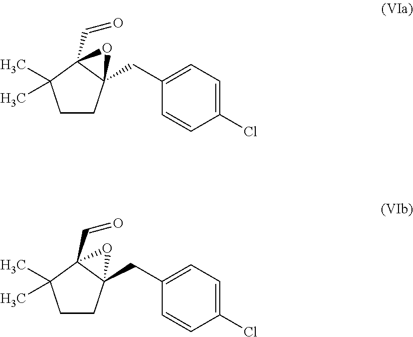 [SMILES: CC1(C)CC[C@]2(Cc3ccc(Cl)cc3)O[C@]12C=O ; CC1(C)CC[C@@]2(Cc3ccc(Cl)cc3)O[C@@]12C=O]
(c′) reducing the epoxy aldehyde of the formula (VIa) or (VIb), or a mixture thereof, with a reducing agent to obtain an epoxy alcohol of the formula (Va) or (Vb), or a mixture thereof.

28. The process according to claim 27, wherein the reducing agent in step (c′) is an alkali metal borohydride.

29. The process of claim 23, where the step (a) of providing an aldehyde of the formula III comprises the isomerization of the epoxide of the formula (II) by treatment with an acid

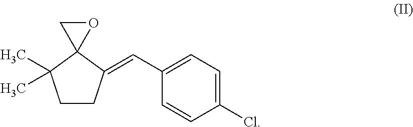 [SMILES: CC1(C)CC/C(=C\c2ccc(Cl)cc2)C12CO2]

30. The process of claim 29, wherein the isomerization is carried out in the presence of an aqueous solution of hydrochloric acid having a concentration of 20 to 37% by weight.

31. The process of claim 29, wherein the solvent or solvent mixture in which the isomerization of step (a) is carried out is not, or is only partly, removed before the reduction of step (b) is initiated, provided the process comprises the step (b).

32. A process for preparing cis-metconazole, which comprises:
(i) providing the diol of the formulae (Ia) or (Ib), or a mixture thereof, by a process of claim 18;
(ii) converting the diol of the formula (Ia) or (Ib), or a mixture thereof, into a compound of the formula (VIIa) or (VIIb), or a mixture thereof, 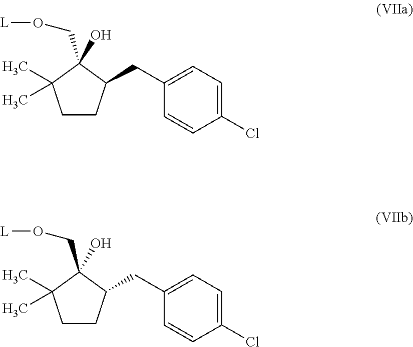
in which L is a leaving group selected from the group of the formula SO₂—R, where R is C₁-C₄-alkyl, C₁-C₄-haloalkyl or phenyl which is unsubstituted or carries 1, 2 or 3 radicals selected from C₁-C₄-alkyl; and
(iii) reacting the compound of the formula (VIIa) or (VIIb), or a mixture thereof with a triazole compound of formula (VIII), 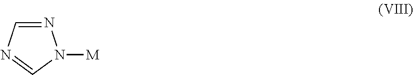
in which M is an alkali metal, in the presence of a base;
or, alternatively, comprising the following steps:
(i) providing the diol of the formulae (Ia) or (Ib), or a mixture thereof, by a process of claim 18;
(ii′) converting the diol of the formula (Ia) or (Ib), or a mixture thereof, into an epoxide of the formula (IXa) or (IXb), or a mixture thereof, in the presence of a base; and 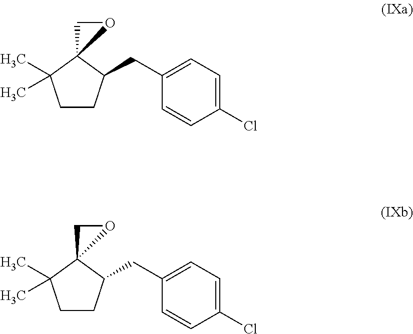 [SMILES: CC1(C)CC[C@H](Cc2ccc(Cl)cc2)[C@@]12CO2 ; CC1(C)CC[C@@H](Cc2ccc(Cl)cc2)[C@]12CO2]
(iii′) reacting the a compound of the formula (IXa) or (IXb), or a mixture thereof with a triazole compound of formula (VIII).

33. The process according to claim 32, wherein (−)-cis-metconazole is selectively prepared by employing the diol of the formula (Ia) in step (ii) or step (ii′) of the process.

34. A compound, which is selected from the group consisting of the aldehyde of the formula (III), 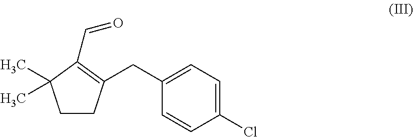 [SMILES: CC1(C)CCC(Cc2ccc(Cl)cc2)=C1C=O]
the epoxy aldehydes of the formulae (VIa) or (VIb), 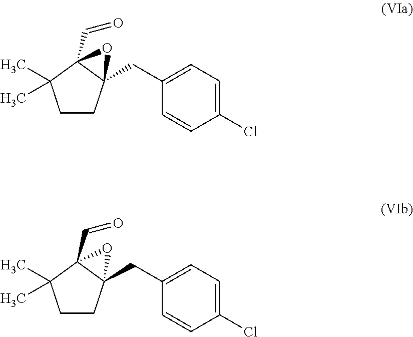 [SMILES: CC1(C)CC[C@]2(Cc3ccc(Cl)cc3)O[C@]12C=O ; CC1(C)CC[C@@]2(Cc3ccc(Cl)cc3)O[C@@]12C=O]
and mixtures of the epoxy aldehyde of the formula (VIa) with the epoxy aldehyde of the formula (VIb).
